(19) United States
(12) Patent Application Publication
(10) Pub. No.: US 2025/0064399 A1
(43) Pub. Date: Feb. 27, 2025

(54) WEARABLE BIOSENSING DEVICE WITH SHIELDING COMPONENT

(71) Applicant: Alio, Inc, Broomfield, CO (US)

(72) Inventors: Jivko Mihaylov, San Jose, CA (US); Samir Shreim, Irvine, CA (US); Anthony F. Flannery, JR., San Diego, CA (US)

(21) Appl. No.: 18/453,194

(22) Filed: Aug. 21, 2023

(51) Int. Cl. A61B 5/00; A61B 5/0295

(52) U.S. Cl. CPC A61B 5/6832; A61B 5/0295; A61B 2562/0204; A61B 2562/0271; A61B 2562/16; A61B 2562/18

(57) ABSTRACT

A wearable biosensing device features a first housing, biosensing logic, a shielding component and a second housing. The biosensing logic includes a sensing assembly positioned within the first housing. Positioned adjacent to the sensing assembly, the shielding component includes a plurality of openings that are arranged to allow for light emitted from or received by components of the sensing assembly. The second housing is coupled to the first housing, and includes an opening to accommodate the shielding component.

### FIELD

Embodiments of the disclosure relate to the field of wearable biosensing devices. More specifically, one embodiment of the disclosure relates to a biosensing device that is mounted to a portion of the body using an adhesive and features a shielding component to avoid leakage of light during a detection phase.

### GENERAL BACKGROUND

The following description includes information that may be useful in understanding the described invention. It is not an admission that any of the information provided herein is prior art or relevant to the presently claimed invention, or that any publication specifically or implicitly referenced is prior art.

Over the last decade, there has been an increasing number of wearable biosensing devices. These devices include one or more biosensors, which collect health-related data from a user. Most of these wearable biosensing devices include a display (e.g., smart watches, Fitbits®, etc.) and are mounted on the user's wrist using a band which encircles the wrist. However, these display-based devices are costly, non-disposable, and cannot be targeted to monitor certain health characteristics, such as blood flow for example, at other location on the patient besides the wrist area.

Adhesive-based wearable biosensing devices tend to be disposable. Their reliability in data collection may be influenced by the amount of sourced light that is accidentally detected by a photodiode. The presence of “leaked” sourced light is quite problematic, especially for photo-plethysmograph (PPG) sensors, because PPG sensors deploy photodiodes responsible for detecting light reflected from vessels under the patient's skin in close proximity to the light sources. When light is leaked from the source to the photodiode, without traveling through the vessel, the analytic results produced from the detected light are incorrect. This may cause erred treatment.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1 is a perspective, anterior-facing view of an exemplary embodiment of a wearable biosensing device.

FIG. 2 is an exploded view of an exemplary embodiment of the wearable biosensing device of FIG. 1, which includes a first housing, a shielding component positioned under biosensing logic, a second housing, and an adhesive layer.

FIG. 3 is an exemplary block diagram of the biosensing logic of FIG. 2.

FIG. 4A is a bottom, posterior-facing view of the biosensing logic inclusive of the sensing assembly of FIG. 3.

FIG. 4B is a more detail posterior-facing view of the sensing assembly implemented as part of the biosensing logic of FIG. 4A.

FIG. 5 is a posterior-facing view of an embodiment of the shielding component of FIGS. 2 & 4B positioned over the sensing assembly.

FIG. 6 is a bottom, posterior-facing view of an exemplary embodiment of the wearable biosensing device of FIG. 1.

### DETAILED DESCRIPTION

Embodiments of the present disclosure generally relate to a wearable biosensing device, which features biosensing logic deployed within a housing that is attached to a patient (wearer) through an adhesive. The biosensing logic includes an electronics assembly, a power assembly, and a sensing assembly positioned between the electronics assembly and the power assembly. Herein, according to one embodiment of the disclosure, the sensing assembly includes a substrate (e.g., printed circuit board) with components mounted thereon. For example, the mounted components may include, but are not limited or restricted to (i) a thermal sensing component (e.g., temperature sensor, etc.), (ii) an audio sensing component (e.g., microphone, etc.) and (iii) a plurality of plethysmograph (PPG) sensors, all of which are positioned on a first (posterior) surface of the substrate.

Positioned adjacent to the sensing assembly, a shielding component includes a plurality of openings. Each opening is configured as a dedicated vertical channel (or conduit) for alignment with (1) the thermal sensing component, (2) the audio sensing component, and (3) each sensing elements associated with a PPG sensor. Therefore, the shielding component is adapted to provide shielding for the gathering of thermal, acoustic, and light-based data. According to one embodiment of the disclosure, the wearable biosensing device is particularly advantageous for monitoring characteristics and operability of a vessel (e.g., an artery, a vein, or an arteriovenous (AV) fistula) by detecting light sourced by a sensing element of the PPG sensor after reflection from and/or refraction through the vessel.

According to one embodiment of the disclosure, the detected light corresponds to collected information associated with physiological properties of that vessel and/or the biological fluid propagating therethrough (e.g., flow, fluid composition, etc.). This information may be useful in monitoring the health of a patient, especially dialysis patients, and may be used to generate an alert signifying a detected health event that is being (or could be) experienced by the wearer of the wearable biosensing device. The biosensing logic may include other sensors (e.g., accelerometer, optical, bioimpedance, electrocardiography etc.), where the sensing elements are configured, alone or in cooperation, to detect/monitor a physiological property and convert the monitored property into an electrical signal, which is subsequently converted to a data representation of the monitored property indicated by the electrical signal. The data representation enables remote monitoring.

As an illustrative example, the wearable biosensing device (with remote monitoring) may be mounted over or proximate to a vessel, such as an artery, a vein, or an arteriovenous (AV) fistula as disclosed in U.S. Pat. Nos. 11,045,123 and 11,406,274, the contents of both of which are incorporated by reference herein. The AV fistula is a surgical connection made between an artery and a vein, usually created by a vascular specialist. The AV fistula facilitates more efficient dialysis than a “line” port due to quicker blood flow during a dialysis session. Normally, the AV fistula is typically located in your arm, however, if necessary, it can be placed in the leg. Other uses for the wearable biosensing device include mounting on the chest for monitoring cardiac functions or on the abdomen for prenatal or intestinal monitoring.

Herein, the shielding component includes a plurality of regions, including a first shield region, a second shield region, and a third shield region. The first shield region includes a cut-out area and a first opening. The cut-out area is sized to accommodate (receive) and retain the thermal sensing component, where a thermal pad associated with the thermal sensing component partially extends from the first cut-out area. The first cut-out area allows for heat conductive contact between the thermal pad and the skin of the user.

The first opening may be configured as a conduit to allow for audio (e.g., sound waves) to propagate to the audio sensing component. Upon receipt of the audio featuring an audio pattern, the wearable biosensing device may be configured to perform certain actions in response to detection of a particular audio pattern. For example, certain detected audio frequencies and/or audio patterns may be used to identify a change in operability (e.g., flow rate, occlusion of the vessel, etc.) experienced by the wearable biosensing device.

The shielding component further includes the second shield region and the third shield region. The second shield region includes openings (e.g., conduits) for sensing elements of a first PPG sensor. Similarly, the third shield region includes openings for sensing elements of a second PPG sensor as described below. The openings for the sensing elements within the second shield region are separated by lateral surface areas, where each lateral surface area has a lesser width than a vertical surface area created between the second shield region and the third shield region.

### I. Terminology

In the following description, certain terminology is used to describe aspects of the invention. The terms “logic” or “assembly” are representative of hardware, firmware, and/or software that is configured to perform one or more functions. As hardware, the logic (or assembly) may include circuitry associated with data processing, data storage and/or data communications. Examples of such circuitry may include, but are not limited or restricted to a processor, a programmable gate array, a microcontroller, an application specific integrated circuit, wireless receiver, transmitter and/or transceiver circuitry, sensors, semiconductor memory, and/or combinatorial logic.

Alternatively, or in combination with the hardware circuitry described above, the logic (or assembly) may include software in the form of one or more software modules (hereinafter, “software module(s)”), which may be configured to support certain functionality upon execution by data processing circuitry. For instance, a software module may constitute an executable applications, daemon application, application programming interface (API), subroutine, function, procedure, applet, servlet, routine, source code, shared library/dynamic load library, or one or more instructions. The “software module(s)” may be stored in any type of a suitable non-transitory storage medium, or transitory storage medium (e.g., electrical, optical, acoustical, or other form of propagated signals such as carrier waves, infrared signals, or digital signals). Examples of non-transitory storage medium may include, but are not limited or restricted to a programmable circuit; a semiconductor memory; non-persistent storage such as volatile memory (e.g., any type of random access memory “RAM”); persistent storage such as non-volatile memory (e.g., read-only memory “ROM”, power-backed RAM, flash memory, phase-change memory, etc.), a solid-state drive, a hard disk drive, an optical disc drive, a portable memory device, or cloud-based storage (e.g., AWS S3 storage, etc. As firmware, the logic (or assembly) may be stored in persistent storage.

The terms “member” and “element” may be construed as a hardware-based logic. The term “attach” and other tenses of the term (e.g., attached, attaching, etc.) may be construed as physically connecting a first member to a second member.

The term “interconnect” may be construed as a physical or logical communication path between two or more components such as a pair of assemblies. For instance, as a physical communication path, wired interconnects in the form of electrical wiring, optical fiber, cable, and/or bus trace. As a logical communication path, the interconnect may be a wireless channel using short range signaling (e.g., Bluetooth™) or longer range signaling (e.g., infrared, radio frequency “RF” or the like).

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

As this invention is susceptible to embodiments of many different forms, it is intended that the present disclosure is to be considered as an example of the principles of the invention and not intended to limit the invention to the specific embodiments shown and described.

### II. Device Attachment Scheme

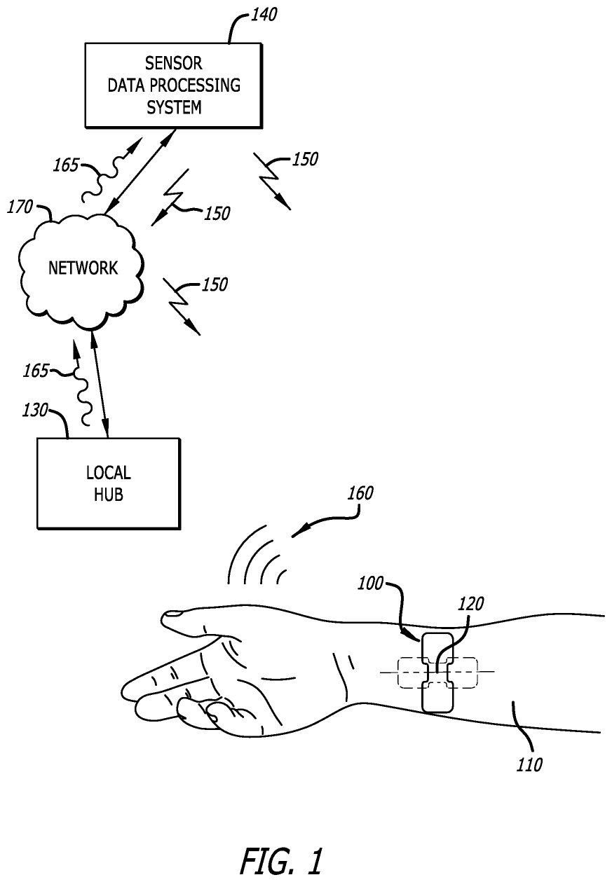

Referring to FIG. 1, a perspective view of an exemplary wearable biosensing device 100 attached to a patient's arm 110 is shown. The wearable biosensing device 100 is intended to be worn over a vessel 120 (e.g., an artery, a vein, or an arteriovenous (AV) fistula) and configured to direct collected information to a remotely located, sensory data processing system 140. For this embodiment, the wearable biosensing device 100 is configured to monitor properties (e.g., characteristics and operability) of the AV fistula 120 by collecting information associated with the AV fistula 120 and the biological fluid propagating therethrough (e.g., flow, fluid composition inclusive of hemoglobin “Hgb” levels, etc.). The collected information may be used for remote monitoring, where the sensory data processing system 140 is configured to determine if a health event exists that warrants generation and transmission of an alert 150 to the patient or an individual involved with the care of the patient.

More specifically, the remote monitoring may involve transmission of the collected information to a local hub 130. The “local hub” 130 constitutes logic (e.g., a device, an application, etc.) that converts a first data representation 160 of the collected information provided in accordance with a first transmission protocol (e.g., Bluetooth™ or other short distance (wireless) transmission protocol) into a second data representation 165. The second data representation 165 may be provided in accordance with a second transmission protocol (e.g., cellular, WiFi™, or other long distance (wireless) transmission protocol) and routes the second data representation 165 to the sensory data processing system 140. The sensory data processing system 140 may include an alert system (not shown), which generates and sends the alert (notification) 150 upon detecting an occurring (or potential) health event that requires attention by a doctor and/or another specified person (including the patient) responsible for addressing any occurring (or potential) health event. The alert 150 may be sent via network 170 for notification over a monitored website or may be sent from the sensor data processing system 140 as an electronic mail (e-mail) message, a text message, or any other signaling mechanism.

### III. General Device Architecture

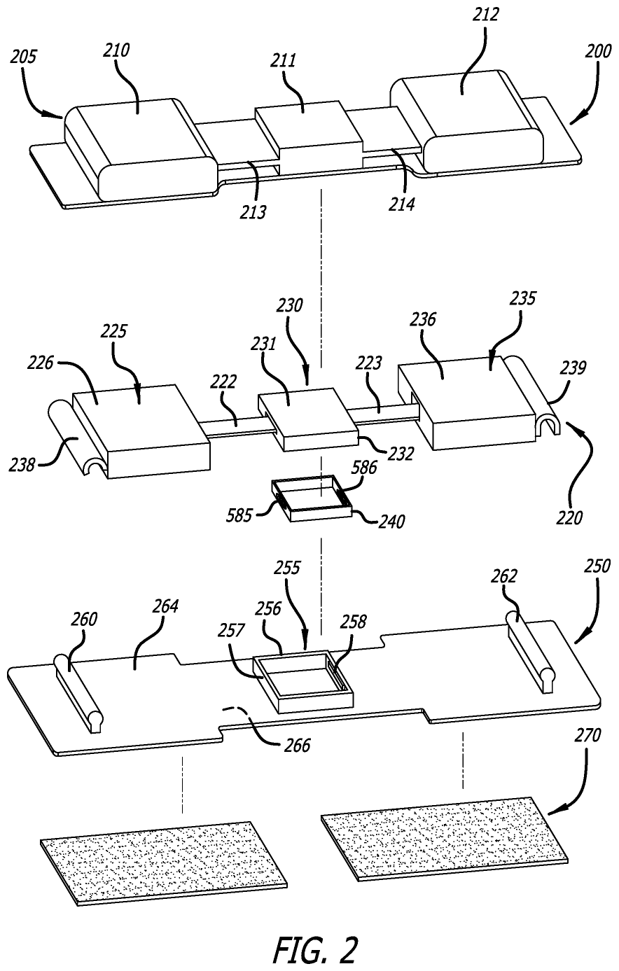

Referring to FIG. 2, an exploded view of an exemplary embodiment of the wearable biosensing device 100 is shown. For this embodiment, the wearable biosensing device 100 includes a first (top) housing 200, packaged biosensing logic 220, a shielding component 240, a second (bottom) housing 250, and an adhesive layer 270. The first housing 200 features multiple (e.g., two or more) lobes 205 formed as part of the first housing 200. The first housing 200 may be made of a flexible, water-impervious material (e.g., a polymer such as silicone, plastic, etc.) through a molding process, where the lobes 205 provide internal chambers for housing the biosensing logic 220.

As an illustrative example, according to one embodiment of the disclosure, the lobes 205 are positioned in a linear orientation, with a first plurality of lobes (e.g., first, second and third lobes 210-212) interconnected by a second plurality of lobes (e.g., fourth and fifth lobes 213-214). The fourth and fifth lobes 213-214 are configured to house interconnects 222 and 223, which provide electrical connections between an electronics assembly 225, a sensing assembly 230, and a power assembly 235 of the biosensing logic 220. As shown, each of the assemblies, namely the electronic assembly 225, the sensing assembly 230, and the power assembly 235, are maintained within a protective package 226, 231 and 236, respectively.

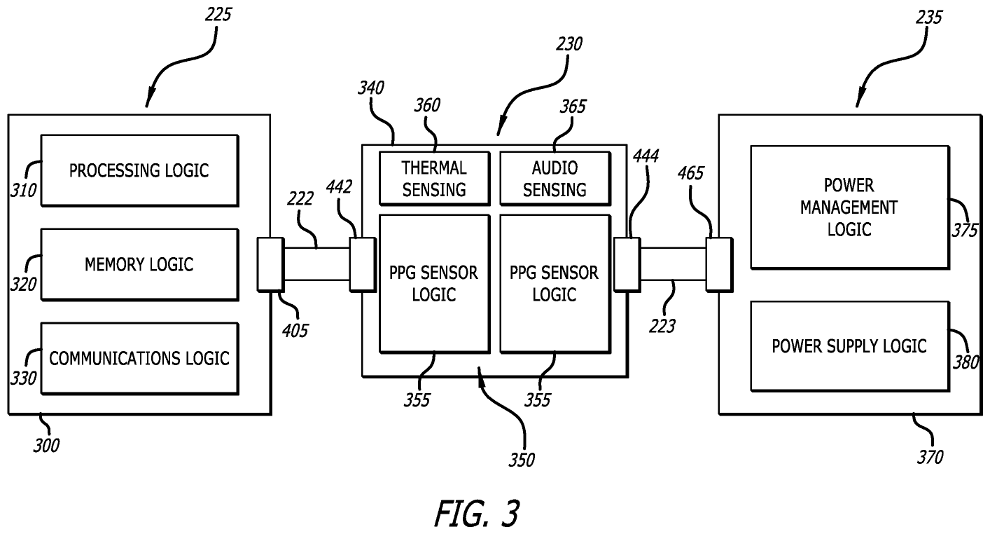

According to one embodiment of the disclosure, housed within the first lobe 210 as shown in FIGS. 2-3, the electronics assembly 225 includes a substrate 300, processing logic 310, memory logic 320, and communications logic 330. Collectively, logic of the electronics assembly 225 is configured to (i) conduct analytics on data gathered by the sensing assembly 230, (ii) store the data (raw) and/or analytic results from the data, and (iii) communicate, via a wireless or a wired connection, the raw and/or the analytic results to a device (e.g., local hub 130 of FIG. 1) remotely located from the wearable biosensing device 100. For example, the electronics assembly 225 may be adapted to transit the first data representation 160 of the collected information to the hub device 130 as shown in FIG. 1.

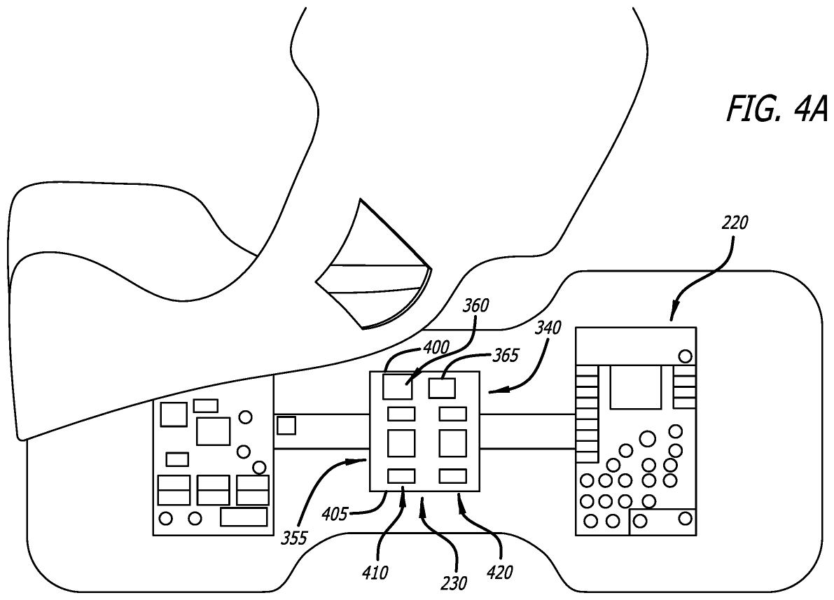

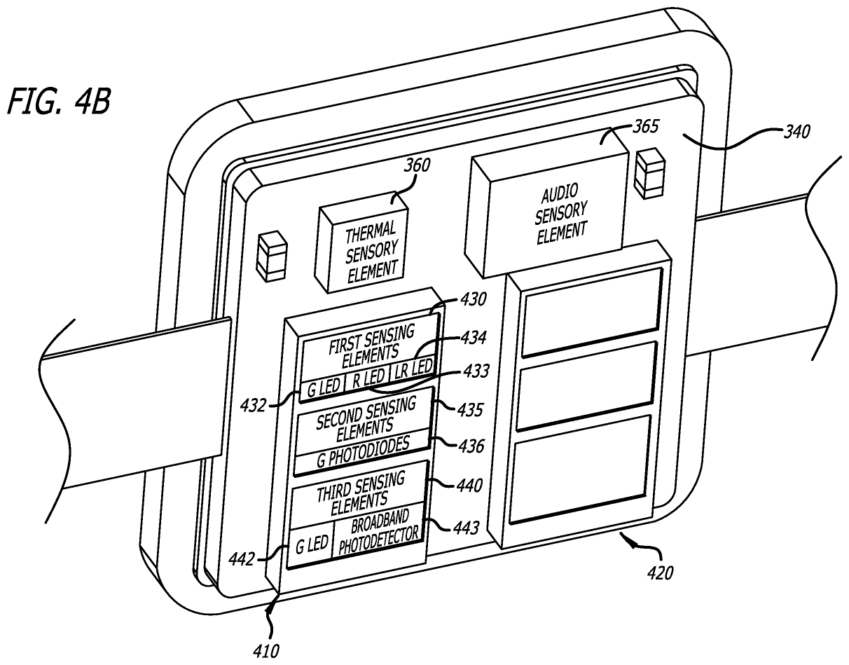

As further shown in FIGS. 2-3, the sensing assembly 230 is housed within the second lobe 211 of the first housing 200. The sensing assembly 230 includes a substrate 340 and one or more sensors 350 (hereinafter, “sensor(s)”) mounted on a posterior surface 345 of the substrate 340. The sensor(s) 350 may include one or more optical sensors configured to emit light and/or detect reflected or refracted light. The optical sensors 350 may include a plurality of photo-plethysmograph (PPG) sensors 355, where each of the plurality of PPG sensors 355 includes multiple light sourcing elements and multiple light detecting elements as illustrated in FIGS. 4A-4B and described below.

Referring still to FIGS. 2-3, coupled to the electronics assembly 225 and the power assembly 235 via interconnects 222 and 223, the sensing assembly 230 may be mounted on or positioned proximate to the vessel 120 of FIG. 1. As a result, the optical sensors 350 may be used to obtain different measurements of properties of the vessel 120 or biological fluid characteristics within the vessel 120 and provide this data to the electronics assembly 225 for analysis. The optical sensors 350 may be arranged in a linear arrangement (as shown) or a circular arrangement with a light sourcing member being positioned centrally and light detecting members distributed radially from the central light sourcing member. Besides the optical sensors 350, the sensing assembly 230 may be configured to include a thermal sensing component 360 (e.g., temperature sensor, etc.) and an audio sensing component 365 (e.g., microphone, etc.). The optical sensors 350 are positioned to emit or detect light via the shielding component 240 as described below.

The power assembly 235 includes a substrate 370, power management logic 375, and power supply logic 380. The power supply logic 380 is configured to provide power to both the components within the sensing assembly 230 as well as the electronics assembly 225. The power management logic 375 is configured to control the distribution of power (e.g., amount, intermittent release, or duration), including disabling of power when the wearable biosensing device 100 is not installed or detached to the wearer to avoid false data collection. The substrate 340 of the sensing assembly 230 may include hardwired traces (power layers) for routing of power from the power supply assembly 235 to components of the sensing assembly 230 and/or components of the electronics assembly 225.

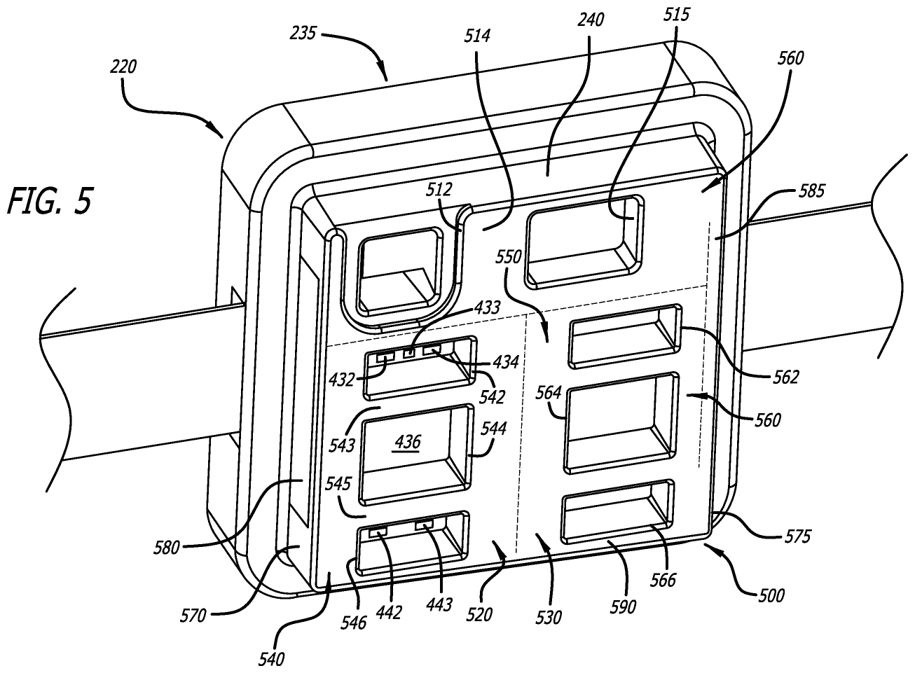

Referring back to FIG. 2, the second housing 250 is configured with a centralized, raised opening 255 that is sized to surround a perimeter of the shielding component 240. Herein, according to one embodiment of the disclosure, a top surface 256 of the raised opening 255 is positioned adjacent to a bottom surface 232 of the protective package 231 for the sensing assembly 230. The raised opening 255 may further include lateral flanges 257-258, which are sized to reside within complementary lateral recesses 585-586 within the shielding component 240 (see also FIG. 5).

As an optional feature, the second housing 250 may include a first raised fastening element 260 and a second raised fastening element 262. These raised fastening elements 260 and 262 are formed on a top (anterior-facing) side 264 of the second housing 250 for attachments to complementary fastening elements 238 and 239 positioned on outer edges of the protective packages 226 and 236, respectively. As shown, the raised fastening elements 260 and 262 are inserted into and secured by fastening elements 238 and 239, and upon applying sufficient forces, the raised fastening elements 260 and 262 may be removed from the fastening elements 238 and 239. As a result, the first housing 200 and the second housing 250 substantially encapsulate the protective packages 226 and 236 while providing partial encapsulation of the protective package 231 inclusive of the sensing assembly 230.

Additionally, the adhesive layer 270 is applied to at least a portion of a bottom surface 266 of the second housing 250. The adhesive layer 270 is adapted to attach to a surface of a patient's skin and remain attached thereto. Alternatively, the adhesive layer 270 may include multiple layers for replacement of the second housing 250 without replacing the packaged biosensing logic 220.

In accordance with another embodiment of the disclosure, in lieu of the fastening elements 238 and 239 in combination with the raised fastening elements 260 and 262, the plurality of magnets (not shown) may be positioned within the second housing 250. These magnets may establish a magnetic coupling to metal fastening elements (e.g., metal connection points) positioned under the power assembly 235 and/or electronics assembly 225 fand/or positioned at ends of the protective packages 226 and 236. Alternatively, the magnets may be positioned as part of the biosensing logic 220 and accessible to metal fastening elements positioned on the second housing 250.

Referring to FIG. 4A, a bottom, posterior-facing view of an exemplary embodiment of the biosensing logic 220 inclusive of the sensing assembly 235 of FIGS. 2-3 is shown. Herein, the sensing assembly 230 features the thermal sensing component 360 and the audio sensing component 365, both of which are positioned proximate to a first edge 400 of the substrate 340. The PPG sensors 355, namely a first PPG sensor 410 and a second PPG sensor 420, are positioned proximate to a second edge 405 of the substate 340, where the second edge 405 is positioned on an opposite side of the substrate 340 than the first edge 400. More specifically, the thermal sensing component 360 and the audio sensing component 365 are interposed between the first edge 400 of the substrate 340 and the PPG sensors 355 while the PPG sensors 355 are interposed between the second edge 405 of the substrate 340 and the thermal sensing component 360 and the audio sensing component 365.

As an illustrative example, each of the PPG sensors 355 (e.g., the first PPG sensor 410) includes one or more light sourcing elements. Herein, the light sourcing elements may include light emitting diodes (LEDs) of different wavelength ranges ranging from 500 nanometers (nm) to 1500 nm. For example, one or more LEDs emitting light with wavelengths ranging between 520-540 nanometers (nm) (e.g., green LED with light emissions of approximately 532 nm), one or more LEDs emitting light with wavelengths ranging between 645-665 nm (e.g., red LED with light emissions of approximately 655 nm), and one or more LEDs emitting light with wavelengths ranging between 930-950 nm (e.g., infrared “IR” LED with light emissions of approximately 940 nm). Besides the light sourcing elements, the sensing assembly 230 further includes light detecting elements, which may include one or more photodiodes (photodetectors) configured to capture reflected or refracted light emitted from a light sourcing element after traveling across an optical path with passage to or through the vessel.

Referring now to FIG. 4B, a more detailed posterior-facing view of the sensing assembly 235 implemented as part of the biosensing logic 220 of FIG. 4A is shown. According to this embodiment of the disclosure, the first PPG sensor 410 includes a first group of sensing elements 430, a second group of sensing elements 435, and a third group of sensing elements 440. The first group of sensing elements 430 includes a green LED 432, a red LED 433, and an IR LED 434. These LEDs 432-434 are grouped together and positioned at a first end portion of the first PPG sensor 410. Although not shown, an accelerometer may be installed as part of the sensing assembly 230.

Herein, each of the LEDs 432-434 is selected to emit light at a wavelength corresponding to the biological metrics to be measured. For example, hematocrit (Hct) may be measured using infrared (IR) light produced by IR LED 434. Some measurements, such as, for example, oxygen saturation, may be measured using optical measurements from red and IR light produced by red LED 433 and IR LED 434. As a result, the type of LEDs (as determined by the emission wavelengths) may be selected based on the interaction of the light and properties of the tissue for the metric being measured. The arrangement of the LEDs is intended to provide multiple source detector combinations to assess the tissue using multiple optical paths for each wavelength.

The second group of sensing elements 435 includes green optimized photodetector 436 while the third group of sensing elements 440 includes a green LED 442 and a broadband photodetector 443. According to this embodiment of the disclosure, the broadband photodetector 443 is positioned at a second end portion of the PPG sensor 410, opposite from the first end portion. The additional distance between the light sourcing elements (red LED 433 and/or IR LED) and the light detecting element (e.g., broadband photodetector 443) is designed to increase an optical path length for light emitted from the red LED 433 and/or IR LED 434 and detected by the broadband photodetector 443. This increased optical path length measures vessels other than superficial vessels immediately under the skin and avoids reflectance error caused by leaked light from these LEDs 433 or 434. In contrast, the green optimized photodetector 436 are interposed between the green LED 432 and green LED 442, and thus, the optical path length for the green emitted light is intended to be shorter to capture a greater number of photons as attenuation due to perfused tissue is substantially greater for green as compared to red/NIR frequency ranges.

The second PPG sensor 420 features the same LED/photodiode architecture as the first PPG sensor 410. The second PPG sensor 420 may operate concurrently with the first PPG sensor 410 or may operate at different time periods. The operability of the PPG sensors 355 are controlled by the electronics assembly 225 in which control signaling is provided through interconnect 222 and/or logic deployed as part of the sensing assembly 230. Although not shown, the PPG sensors 355 may be communicatively coupled to amplifiers for use in amplifying the analytic results. The analytic results may be utilized to understand and monitor fluid flow, vessel characteristics (e.g., depth, diameter, etc.), oxygenation levels of fluid flowing through the vessel, hemoglobin levels, and other metrics to identify whether a status of the health of the patient.

Referring to FIG. 5, a posterior-facing view of an embodiment of the shielding component 240 of FIGS. 2-3 positioned over the sensing assembly 235 of the biosensing logic 220 is shown. Herein, the shielding component 240 operates as shielding for components associated with temperature sensing, acoustic (audio) sensing, and light sensing. According to one embodiment of the disclosure, the shielding component 240 is made of a thermoplastic polymer such as acrylonitrile butadiene styrene (ABS) material with a pigment additive that is highly absorptive of IR light (e.g., light frequency exceeding 700 nm). One example of the pigment-ABS material is ABS Toyolac 700 x01 by Toray Plastics of Malaysia. Other types of high absorptive ABS material may include ABS embedded with carbon or carbon fibers. According to another embodiment of the disclosure, the shielding component 240 may be made of a deformable, pliable material configured to confirm to tissue surrounding the wearable biosensing device 100 to present gaps between the tissue and an outer surface of the shielding component 240. Yet another embodiment of the disclosure, the shielding component 240 may be made of a carbon embedded ABS material such as RTP 600 S-809479 Black.

As further shown in FIG. 5, the shielding component 240 includes a cut-out area 512 and a number of openings positioned to allow for light emitted from or received by components of the sensing assembly. More specifically, the shielding component 240 includes a plurality of regions 500, including a first shield region 510, a second shield region 520, and a third shield region 530. The first shield region 510 includes a cut-out area 512 and a first opening 515. The cut-out area 512 is sized to accommodate (receive) the thermal sensing component 360 (see FIG. 4A) in which a thermal pad 513 extends from or is flush with a top surface 514 of the cut-out area 512 to establish a heat conductive contact a patient's skin to measure temperature of the patient. Alternatively, the thermal pad 513 of the thermal sensing component 360 may reside within the region, and a thermal conduit (e.g., a piece of stainless steel or other metal, nylon, etc.) may be positioned between the thermal sensing component 360 and a tissue of the patient's arm 110 of FIG. 1.

The first shield region 510 further includes the first opening 515, which may be configured as a conduit to allow for sound waves to propagate to the audio sensing component 365 and to detect audio. The detected audio may be processed by the processing logic 310 of the electronics assembly 225, and based on the characteristics of the detected audio matching a particular audio pattern, performing certain actions. In particular, certain detected audio frequencies and/or audio patterns may be used to identify a change in operability (e.g., flow rate, etc.) experienced by the wearable biosensing device. For example, the frequency of the audio may be used to identify flow rates, and therefore, an occlusion of the vessel.

It is contemplated that different selected material may be placed within the air gap of the window 515 to enhance or filter different aspects of the detected audio signal. As an illustrative example, the audio frequency of human speech is relatively higher than the lower frequency of the audio associated with a heartbeat. By selecting a material that operates as a low-pass filter, voice is filtered from audio gathered, and thereby, the signal processing conducted to determine flow rate and/or vessel occlusions that may be assisted with knowledge of the timing of the heartbeat. The material may be deposited as a filling material within the air gap formed by window 515, a membrane laid over the window 515, or the like.

As shown in FIG. 5, the second shield region 520 includes a plurality of openings 540 associated with the different groups of sensing elements 430/435/440 implemented within the first PPG sensor 410. The openings 540, operating as window areas, includes a first window area 542 that provides a conduit for the first group of sensing elements 430, in particular green/red/IR LEDs 432-434. Similarly, a second window area 544 provides a conduit for the second group of sensing elements 435 (e.g., green photodiodes 436) while the third window area 546 provides a conduit for the third group of sensing elements 440 (e.g., green LED 442 and broadband detector 443). The window areas 542/544/546 are sized with a depth that prevents surface reflections, especially surface reflections that could result in light shunting/piping, e.g., light that has not substantially interacted with the targeted tissue section. The width distances of the shielding areas 543 and 545 between window areas 542/544 and 544/546 are narrower than the distance of the shielding area 550 between window areas 542/562.

In order to account for the patient's motion, one of the shielding areas (e.g., area 543) may be removed to allow light from the green photodiodes 436 to be captured by green LED 432. As a result, a majority of the light from the green photodiode 436 would be captured from reflection of the transmitted light from the tissue, where signaling created from the captured reflection light may be used as a noise cancellation signal to account for motion of the patient with the wearable biosensing device 100.

As shown in FIG. 5, the third shield region 530 includes a plurality of window areas 560 associated with the different groups of sensing elements 440 implemented within the second PPG sensor 420. The window areas 560 includes a first window area 562 that provides a conduit for a first group of sensing element, in particular LEDs 442-444. Similarly, the second window area 564 provides a conduit for the second group of sensing elements (green photodiodes) while the third window area 566 provides a conduit for the third group of sensing elements 440 (green LED and broadband photodiode). The architecture of the shielding component 240 within the third shield region 530 is similar to the architecture of the second shield region 520 described above.

Although not shown, it is contemplated that portion of the first shield region 510, the second shield region 520 and/or the third shield region 530 may be configured with acoustic shielding. For example, as one illustrative embodiment, acoustic shielding may be integrated with window areas of the shielding component 240 in addition to other acoustic shielding materials behind the substrate 340. Alternatively, the second housing 250 could be configured with audio dampening material (e.g., mass loaded vinyl). As yet another alternative embodiment, audio deadening material may include an audio deadening material positioned between the second housing 250 and the substrate 340.

The lateral sides 570 and 575 of the shielding component 240 includes recesses 580 and 585, which are sized to engage with the lateral flanges 257-258 of the raised opening 255 positioned on the second housing 250 as shown in FIG. 2. Although not shown, it is contemplated that an optically transparent layer may be applied to a posterior-facing surface 590 of the shielding component 240 to prevent environmental matter and contaminants from lodging within the window areas 515/542/544/546/562/564/566. Examples of this material would be a substantially transparent, yet biocompliant silicone material such as Momentive RTV 118. Alternatively, a hard plastic may be used such as acrylic, polycarbonate, or PPG CR-39.

It is further contemplated that, in accordance with another embodiment of the disclosure, one or more of the gaps formed by window areas 515, 542, 544, 546, 562, 564 or 566 may be filled with a transparent material to allow for light transmission and capture, but prevent build-up of contaminants within the gaps. The transparent material within one or more gaps formed by window areas 515, 542, 544, 546, 562, 564 or 566 may be rounded or filed to generally operate as a lens with additional light capturing capabilities and/or shorten path lengths of the light to increase signal.

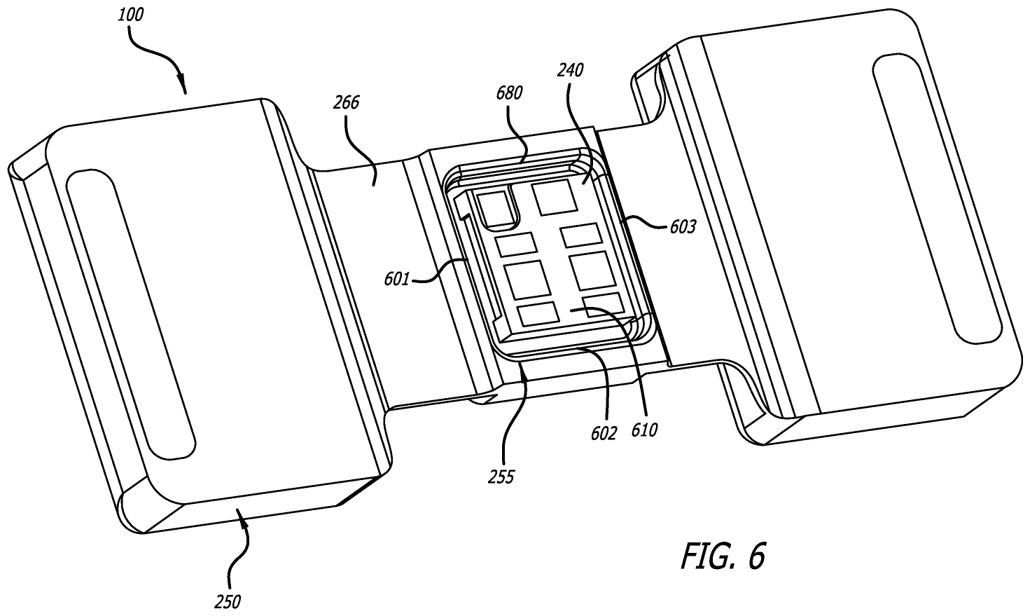

Referring now FIG. 6, a bottom, posterior-facing view of an exemplary embodiment of the wearable biosensing device 100 of FIG. 1 is shown. As shown, the shielding component 240 is positioned to reside within a cavity formed by sidewalls 600-603 of the centralized, raised opening 255. Furthermore, the shielding component 240 is provided a prescribed depth so that a posterior surface 610 of the shielding component 240 is coplanar with the posterior-facing (bottom) surface 266 of the second housing 250. The shielding component 240 may be positioned over the vessel and mitigate detection by PPG sensors of light emitted from the light sourcing elements (e.g., LEDs 432-434 and 442) that is reflected (or leaked) prior to passage through the skin of the patient. By reducing the amount of light leakage, the shielding component 240 may reduce the number and degree of errors in determinations for measured biological metrics. The erred determinations may propagate to errors (false positives and/or false negatives) in initiation of the alerts as described herein.

What is claimed is:

1. A wearable biosensing device, comprising:
a first housing including a plurality of lobes; and
a biosensing logic partially encapsulated within the plurality of lobes, the biosensing logic including a sensing assembly positioned within a first lobe of the plurality of lobes;
a shielding component positioned adjacent to the sensing assembly, the shielding component including a plurality of openings positioned to allow for light emitted from or received by components of the sensing assembly; and
a second housing coupled to the first housing, the second housing including an opening to accommodate the shielding component.

2. The wearable biosensing device of claim 1, wherein the second housing comprises a raised opening sized to surround a perimeter of the shielding component.

3. The wearable biosensing device of claim 2, wherein a top surface of the raised opening is positioned adjacent to a bottom surface of a protective package for the sensing assembly.

4. The wearable biosensing device of claim 2, wherein the raised opening further comprises at least one lateral flange sized to reside within a complementary lateral recesses within the shielding component.

5. The wearable biosensing device of claim 1, wherein the shielding component including a plurality of shield regions including a first shield region, the first shield region includes a cut-out area sized to accommodate a thermal sensing component and an opening sized to accommodate an audio sensing component configured to detect audio and cause the wearable biosensing device to perform certain actions in response to detection of a particular audio pattern.

6. The wearable biosensing device of claim 1, wherein the shielding component comprises a plurality of shield regions including a shield region, the shield region includes a first plurality of openings each associated with one of a corresponding plurality of sensing elements associated with a first plethysmograph (PPG) sensor.

7. The wearable biosensing device of claim 6, wherein the first plurality of openings comprises a first window area that provides a conduit for a first group of sensing elements including a combination of a first green light emitting diode, a first red light emitting diode, and a first infrared light emitting diode.

8. The wearable biosensing device of claim 7, wherein the first plurality of openings further comprises a second window area that provides a conduit for a second group of sensing elements including one or more photodiodes.

9. The wearable biosensing device of claim 8, wherein the first plurality of openings further comprises a third window area that provides a conduit for a third group of sensing elements including at least a second light emitting diode and a broadband detector,

10. The wearable biosensing device of claim 6, wherein the shield region further includes a second plurality of openings each associated with one of a corresponding plurality of sensing elements associated with a second plethysmograph (PPG) sensor.

11. The wearable biosensing device of claim 1, wherein the shielding component comprises a first shield region includes a first plurality of openings each associated with one of a corresponding plurality of sensing elements associated with a first plethysmograph (PPG) sensor, the plurality of sensing elements include one or more optical sensors and one or more photodetectors deployed as part of the sensing assembly to emit and detect light reflecting or refracting from a vessel to detect properties of the vessel and biological fluid flow through the vessel.

12. The wearable biosensing device of claim 11, wherein the vessel corresponds to an arteriovenous (AV) fistula.

13. A wearable biosensing device, comprising:
a first housing including a plurality of lobes; and
a biosensing logic partially including a sensing assembly positioned within a first lobe of the plurality of lobes;
a shielding component; and
a second housing coupled to the first housing, the second housing including an opening to accommodate the shielding component
wherein the shielding component interposed between the sensing assembly and the opening, the shielding component including a plurality of openings positioned to allow for light emitted from or received by components of the sensing assembly.

14. The wearable biosensing device of claim 13, wherein the opening constitutes a raised opening sized to surround a perimeter of the shielding component, a top surface of the raised opening is positioned adjacent to a bottom surface of a protective package for the sensing assembly.

15. The wearable biosensing device of claim 14, wherein the raised opening further comprises at least one lateral flange sized to reside within a complementary lateral recesses within the shielding component.

16. The wearable biosensing device of claim 13, wherein the shielding component including a plurality of shield regions including a first shield region, the first shield region includes a cut-out area sized to accommodate a thermal sensing component and an opening sized to accommodate an audio sensing component configured to detect audio and cause the wearable biosensing device to perform certain actions in response to detection of a particular audio pattern.

17. The wearable biosensing device of claim 13, wherein the shielding component comprises a plurality of shield regions including a first shield region, the first shield region includes a first plurality of openings each associated with one of a corresponding plurality of sensing elements associated with a first plethysmograph (PPG) sensor.

18. The wearable biosensing device of claim 13, wherein the shielding component comprises a first shield region includes a first plurality of openings each associated with one of a corresponding plurality of sensing elements associated with a first plethysmograph (PPG) sensor, the plurality of sensing elements include one or more optical sensors, and one or more photodetectors deployed as part of the sensing assembly to emit and detect light reflecting or refracting from a vessel to detect properties of the vessel and biological fluid flow through the vessel.

19. The wearable biosensing device of claim 18, wherein the vessel corresponds to an arteriovenous (AV) fistula

20. A wearable biosensing device, comprising:
a first housing; and
a biosensing logic partially including a sensing assembly positioned within a first lobe formed within the first housing;
a shielding component; and
a second housing coupled to the first housing, the second housing including an opening to accommodate the shielding component,
wherein the shielding component interposed between the sensing assembly and the opening, the shielding component including a plurality of openings positioned to allow for light emitted from or received by components of the sensing assembly.
